(19) United States
(12) Patent Application Publication
(10) Pub. No.: US 2016/0017427 A1
(43) Pub. Date: Jan. 21, 2016

(54) TREATING FEMALE PELVIC ORGAN PROLAPSE

(71) Applicants: Patrick J. Culligan, Madison, NJ (US); Richard Scott, Far Hills, NJ (US)

(72) Inventors: Patrick J. Culligan, Madison, NJ (US); Richard Scott, Far Hills, NJ (US)

(21) Appl. No.: 14/805,329

(22) Filed: Jul. 21, 2015

Related U.S. Application Data
(60) Provisional application No. 62/027,200, filed on Jul. 21, 2014.

(51) Int. Cl. C12Q 1/68; A61B 10/02

(52) U.S. Cl. CPC C12Q 1/6883; A61B 10/02; A61B 2010/0216; C12Q 2600/156; C12Q 2600/118

(57) ABSTRACT

Provided are methods for diagnosing an increased risk of failure in a female pelvic organ prolapse surgery. Also provided are devices and kits for detection of a SNP associated with increased risk of failure in a female pelvic organ prolapse surgery.

### CROSS-REFERENCE TO RELATED APPLICATIONS

This application claims the benefit of priority to U.S. Provisional Application Serial No. 62/027,200 filed on 21 Jul. 2014, which is incorporated herein by reference in its entirety.

### STATEMENT REGARDING FEDERALLY SPONSORED RESEARCH OR DEVELOPMENT

Not Applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### BACKGROUND OF THE INVENTION

In the United States alone, millions of women suffer from the condition known as pelvic organ prolapse (POP), the prevalence of which can be expected to increase nearly 50% by 2050. POP is understood in the art to be any descent of the anterior vaginal wall (aka cystocele or urethrocele), the vaginal apex (aka uterine or vaginal vault prolapse), or the posterior vaginal wall (aka rectocele, perineocele), or any combination of these. Symptoms that can be commonly associated with POP include pelvic heaviness, vaginal bulging, incomplete bowel or bladder emptying, needing to splint the posterior vaginal wall or perineum to defecate, or discomfort during sexual intercourse. The etiology of POP can be multifactorial or complicated. The main risk factors can be vaginal childbirth, frequent increases in intra-abdominal pressure (such as occurs with heavy lifting or chronic constipation), aging, or genetic predisposition to connective tissue abnormalities. Several studies have reported on variations in the expression of certain genes that could lead to development of pelvic organ prolapse (POP), but no studies to date have reported a genetic basis for failure of an operation to correct POP.

A sacrocolpopexy surgical procedure can involve placement of a bridge of graft material between the prolapsed vagina and the anterior longitudinal ligament of the sacrum. Sacrocolpopexy is widely considered the “gold standard” procedure for correction of prolapse involving the apex with success rates reported between 80-100% depending on the techniques employed and definitions of success used. Originally an open abdominal procedure designed primarily for the correction of recurrent vaginal vault prolapse, the sacrocolpopexy is now often performed via the laparoscopic approach—with or without robotic assistance for virtually any variety of POP—whether or not the patient still has a uterus.

One surgical method for treating POP involves extensive dissection in the vesicovaginal and rectovaginal spaces. A pre-formed polypropylene “Y-mesh” can be attached to the full length and width of the anterior vaginal wall (down to the level of the trigone) and to the full length and width of the posterior vaginal wall (down to the level of the perineal body). Using standardized robotic techniques, the inventors Surgical cure rates using this technique have been reported at 97% at one year, with few failures typically occurring in the distal most anterior or posterior segments.

But a small group of patients can experience early objective overt failures despite having the extensive procedure described above in the early post-operative period. These failures have not been explained by differing surgical techniques, poor adherence to post-operative restrictions, or complicated peri-operative courses. In other words, the failures in this small group were clinically difficult to explain.

SNP genotyping is generally understood to be the measurement of genetic variations of single nucleotide polymorphisms (SNPs) between members of a species. A SNP is understood as a single base pair mutation at a specific locus, usually consisting of two alleles (where the rare allele frequency can be >1%). SNPs can be involved in the etiology of many human diseases and have pharmacogenetic applications. Because SNPs can be conserved during evolution, they can be a marker for use in quantitative trait loci (QTL) analysis or in association studies in place of microsatellites.

### SUMMARY OF THE INVENTION

Among the various aspects of the present disclosure is the provision of a method for diagnosing an increased risk of failure in a female pelvic organ prolapse surgery. In some embodiments, the method includes detecting at least one single nucleotide polymorphism (SNP) in a subject in need; or correlating presence of the SNP with an increased risk of failure in a female pelvic organ prolapse surgery. In some embodiments, the at least one SNP is selected from a single nucleotide variation T at position 26 of SEQ ID NO: 2; a single nucleotide variation C at position 26 of SEQ ID NO: 4; a single nucleotide variation G at position 26 of SEQ ID NO: 6; a single nucleotide variation G at position 26 of SEQ ID NO: 8; a single nucleotide variation A at position 26 of SEQ ID NO: 10; or a single nucleotide variation C at position 26 of SEQ ID NO: 12.

In some embodiments, the SNP is selected from a single nucleotide variation T at position 26 of SEQ ID NO: 2 or a single nucleotide variation A at position 26 of SEQ ID NO: 10. In some embodiments, the method further includes a SNP selected from a single nucleotide variation C at position 26 of SEQ ID NO: 4; a single nucleotide variation G at position 26 of SEQ ID NO: 6; or a single nucleotide variation G at position 26 of SEQ ID NO: 8.

In some embodiments, the SNP is present in a sequence selected from SEQ ID NO: 1 (dbSNP ID rs171821); SEQ ID NO: 3 (dbSNP ID rs1423113); SEQ ID NO: 5 (dbSNP ID rs16877757); SEQ ID NO: 7 (dbSNP ID rs259043); SEQ ID NO: 9 (dbSNP ID rs249038); or SEQ ID NO: 11 (dbSNP ID rs2544600). In some embodiments, the SNP is present in a sequence selected from the group consisting of SEQ ID NO: 1 (dbSNP ID rs171821); or SEQ ID NO: 9 (dbSNP ID rs249038). In some embodiments, the method further includes a SNP present in a sequence selected from the group consisting of: SEQ ID NO: 3 (dbSNP ID rs1423113); SEQ ID NO: 5 (dbSNP ID rs16877757); or SEQ ID NO: 7 (dbSNP ID rs259043).

In some embodiments, detecting the SNP includes detecting a plurality of SNPs.

In some embodiments, the subject has, is diagnosed with, is suspected of having, or is at risk for developing female pelvic organ prolapse.

In some embodiments, the method further includes providing a biological sample from the subject; wherein detecting the SNP includes detecting the SNP in the biological sample.

In some embodiments, the sample includes a buccal swab.

In some embodiments, the method further includes if the SNP is detected, altering a surgical protocol, selecting a therapeutic approach other than surgery, selecting a therapeutic protocol other than sacrocolpopexy; delaying a surgical protocol, or forgoing a surgical protocol.

In some embodiments, the method further includes an exome analysis.

In some embodiments, the SNP is detected by Dynamic allele-specific hybridization, DASH; molecular beacons; SNP microarrays; Restriction fragment length polymorphism, RFLP; tetra-primer ARMS-PCR; Flap endonuclease, FEN; primer extension; Taq DNA polymerase 5′-nuclease activity in a TaqMan assay; oligonucleotide ligation assay; single strand conformation polymorphism; temperature gradient gel electrophoresis; denaturing high performance liquid chromatography; high resolution melting analysis; DNA mismatch-binding proteins; SNPlex; or pyrosequencing.

In some embodiments, the SNP is detected by an array.

In some embodiments, the SNP is detected by an array including: a labeled allele-specific oligonucleotide (ASO) probe specific for (i) a SNP selected from the group consisting of a single nucleotide variation T at position 26 of SEQ ID NO: 2; a single nucleotide variation C at position 26 of SEQ ID NO: 4; a single nucleotide variation G at position 26 of SEQ ID NO: 6; a single nucleotide variation G at position 26 of SEQ ID NO: 8; a single nucleotide variation A at position 26 of SEQ ID NO: 10; or a single nucleotide variation C at position 26 of SEQ ID NO: 12; or (ii) a SNP present in SEQ ID NO: 1 (dbSNP ID rs171821), SEQ ID NO: 3 (dbSNP ID rs1423113), SEQ ID NO: 5 (dbSNP ID rs16877757), SEQ ID NO: 7 (dbSNP ID rs259043), SEQ ID NO: 9 (dbSNP ID rs249038), or SEQ ID NO: 11 (dbSNP ID rs2544600); or a detection system that records or interprets a hybridization signal between the ASO probe or a polynucleotide sequence from a biological sample of the subject.

In some embodiments, the method further includes contacting a biological sample of the subject and the array.

In some embodiments, the method further includes detecting a level of TGFβ in the subject, wherein detecting the decreased level of TGFβ in the subject is associated with increased risk of failure in a female pelvic organ prolapse surgery.

In some embodiments, the method further includes if the SNP is detected, altering a surgical protocol, selecting a therapeutic approach other than surgery, selecting a therapeutic protocol other than sacrocolpopexy; delaying a surgical protocol, forgoing a surgical protocol, or increasing a TGFβ level in the subject.

Another aspect provides a device for detection of a single nucleotide polymorphism (SNP) associated with increased risk of failure in a female pelvic organ prolapse surgery. In some embodiments, the device includes: one or more labeled allele-specific oligonucleotide (ASO) probes specific for (i) a SNP selected from the group consisting of a single nucleotide variation T at position 26 of SEQ ID NO: 2; a single nucleotide variation C at position 26 of SEQ ID NO: 4; a single nucleotide variation G at position 26 of SEQ ID NO: 6; a single nucleotide variation G at position 26 of SEQ ID NO: 8; a single nucleotide variation A at position 26 of SEQ ID NO: 10; and a single nucleotide variation C at position 26 of SEQ ID NO: 12; or (ii) a SNP present in SEQ ID NO: 1 (dbSNP ID rs171821), SEQ ID NO: 3 (dbSNP ID rs1423113), SEQ ID NO: 5 (dbSNP ID rs16877757), SEQ ID NO: 7 (dbSNP ID rs259043), SEQ ID NO: 9 (dbSNP ID rs249038), or SEQ ID NO: 11 (dbSNP ID rs2544600). In some embodiments, the device includes a detection system that records or interprets a hybridization signal between the ASO probe and a polynucleotide sequence from a biological sample of the subject. In some embodiments, the device includes an array, the array comprising a matrix and a plurality of detection spots on or in the matrix, each detection spot comprising a unique ASO probe. In some embodiments, the array device consists essentially of detection spots comprising unique ASO probe specific for a SNP associated with increased risk of failure in a female pelvic organ prolapse surgery. In some embodiments, the array device includes (i) detection spots comprising unique ASO probe specific for a SNP associated with increased risk of failure in a female pelvic organ prolapse surgery or (ii) detection spots not so associated.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a Manhattan plot showing the genome-wide association analyses.

### DETAILED DESCRIPTION OF THE INVENTION

The present disclosure is directed at least in part to a genetic basis for failure of a female pelvic organ prolapse surgery (e.g., sacrocolpopexy). Findings described herein arose at least in part from the goal of determining whether a genetic basis may exist for early female pelvic organ prolapse surgical failures seen in a population of subjects.

Predicting poor surgical outcome among women planning to undergo prolapse surgery, as described herein, has immediate and important implications. Surgical procedures for pelvic organ prolapse can be quite common. In the United States alone, approximately 300,000 women undergo surgery for this condition yearly, and unfortunately up to 30% of these women will require a repeat operation. While many investigators have attempted to find specific genetic variations that might cause pelvic organ prolapse, very little evidence of such a relationship has been found. In fact, a recent systematic review and metaanalysis described only ‘moderate’ epidemiological credibility for the variation of COL1A1 with the development of prolapse. That study also stressed the need for exploration of further variants to not only help explain the complex pathophysiology of prolapse but to also provide methods of prevention or treatment. Even if future studies can identify a specific genetic variation that results in pelvic organ prolapse, the clinical usefulness of such a finding may be limited. In contrast, predicting poor surgical outcome among women planning to undergo prolapse surgery, as described herein, has immediate and important implications.

Endofin

The Endofin gene encodes an endosomal protein that belongs to the FYVE zinc finger family of proteins that is understood to regulate membrane trafficking in the endosome.

Endofin can also facilitates Transforming Growth Factor-beta (TGF-β) as a scaffold protein. Endofin can also promote R-Smad-Smad 4 complex formation. This R-Smad-Smad 4 complex is understood to lead to apoptosis in cells.

In studies described herein, a SNP near the ZFYVE16 gene (also known as Endofin) on chromosome 5 was identified in a group of women who experienced clinically-unusual early overt failure following sacrocolpopexy. 10 subjects having experienced early multi-compartment pelvic organ prolapse (POP) recurrence after robotic sacrocolpopexy were selected, where controls were 40 randomly selected patients with known success 12 months after that same procedure. There were no baseline demographic or clinical differences between the cases and controls. DNA from the cases and controls were isolated from buccal swabs and genotyped on a single nucleotide polymorphism (SNP) array to direct more detailed exome analyses. Exome sequences were mapped to the Human Genome Reference Sequence (GRCh37) and variants were compared between groups and to participants in the 1000 Genomes Project. Statistical analyses included Correlation/Trend test, Cochran-Armitage test, and logistic regression. TaqMan assay was used for verification and p-values were adjusted using the False Discovery Rate (FDR). Demographics of groups were compared using Chi square, Mann Whitney U, and t-tests.

Results reported herein showed a SNP of SEQ ID NO: 1 (rs171821) located near the ZFYVE16 gene was associated with the sacrocolpopexy failure group but not the controls (with correlation/trend test on the basic allele model with an FDR-adjusted p-value of 0.046). Exome analyses of this gene yielded another SNP of SEQ ID NO: 9 (rs249038 (G/A)), in 6 out of 10 cases, and none of the controls, (p=0.02). This SNP causes a heterozygous missense mutation of glycine to serine predicted to be deleterious by PROVEAN (Protein Variation Effect Analyzer), and was also very rare among participants in the 1000 Genomes Project (p<0.001).

As described herein, two SNPs located near the ZFYVE16 gene (also known as Endofin) on chromosome 5 has been shown to be present among a group of female subjects known to have experienced clinically unusual early overt multi compartment sacrocolpopexy failure (but not in controls). Accordingly, a candidate gene, Endofin, can be linked to early recurrence of genital prolapse.

Studies described herein include the use of a standardized surgical technique for all cases and controls by two surgeons at a single center. Both surgeons were beyond the robotic learning curve at the study outset. As described herein, the studies use a systematic approach in identifying the cases. Every effort was made to collect only true clinical outliers as cases—thus holding to the concept of extreme phenotype analyses. Furthermore, controls were properly selected at random (using a random number generator) in a 4:1 ratio from a group of similar patients with known surgical success during the same period of time.

TGF-B

Endofin facilitates Transforming Growth Factor-beta (TGF-β) as a scaffold protein. TGF-β is a protein that controls proliferation and cellular differentiation. TGF-β is a cytokine that can be involved in immunity, cancer, asthma, diabetes, heart disease, hereditary telangiectasia, Parkinson's, AIDS, Marfan's disease, Ehlers Danlos syndrome, or Loey-Ditz Syndrome. TGF-β can play an important role in growth and development, inflammation and repair, or host immunity, as TGF-β can control fibroblast proliferation, cellular differentiation, or promote collagen synthesis by increasing the extracellular matrix production. TGF-β levels can be relatively high within tissue undergoing wound healing or remodeling.

Alterations in TGF-β can be associated with connective tissue disorders such as Marfan syndrome or Loey-Ditz Syndrome. Furthermore, women with these connective tissue disorders can have relatively high rates of urinary incontinence and pelvic organ prolapse. Qi et al demonstrated amongst a group of women with prolapse that the expression of TGF-β1 protein was significantly lower than that of a control group without prolapse. As such, TGF-β1 expression can be lower in groups of women with prolapse compared to a control group without prolapse.

SNPs

A SNP described herein can be used as a tool to identify a subject that can have an increased probability of recurrence after female pelvic organ prolapse surgery. Such diagnostic can provide an individualized treatment plan or avoid multiple surgeries. For example, when a SNP described herein is identified, a subject can choose a therapeutic approach other than surgery, delay a surgical approach, or forgo a surgical approach.

SNPs described herein can be used as a diagnostic to identify a subset of female subjects at increased risk for early overt failure following female pelvic organ prolapse surgery.

A SNP can be present in present in dbSNP ID rs171821 (SEQ ID NO: 1). The dbSNP ID rs171821 (SEQ ID NO: 1) has a single nucleotide variation Tin place of ancestral C at position 26 (C26T) of SEQ ID NO: 2 (ancestral sequence) from Chromosome 5 of Homo sapiens.

A SNP can be present in dbSNP ID rs1423113 (SEQ ID NO: 3). The dbSNP ID rs1423113 (SEQ ID NO: 3) has a single nucleotide variation C in place of ancestral A at position 26 (A26C) of SEQ ID NO: 4 (ancestral sequence) from Chromosome 5 of Homo sapiens.

A SNP can be present in dbSNP ID rs16877757 (SEQ ID NO: 5). The dbSNP ID rs16877757 (SEQ ID NO: 5) has a single nucleotide variation G in place of ancestral A at position 26 (A26C) of SEQ ID NO: 6 (ancestral sequence) from Chromosome 5 of Homo sapiens.

A SNP can be present in dbSNP ID rs259043 (SEQ ID NO: 7). The dbSNP ID rs259043 (SEQ ID NO: 7) has a single nucleotide variation G in place of ancestral T (reverse strand, nucleotide order in database is SNP/ancestral) at position 26 (T26G) of SEQ ID NO: 8 (ancestral sequence) from Chromosome 5 of Homo sapiens.

A SNP can be present in dbSNP ID rs249038 (SEQ ID NO: 9). The dbSNP ID rs249038 (SEQ ID NO: 9) has a single nucleotide variation A in place of ancestral G at position 26 (G26A) of SEQ ID NO: 10 (ancestral sequence) from Chromosome 5 of Homo sapiens.

A SNP can be present in dbSNP ID rs2544600 (SEQ ID NO: 11). The dbSNP ID rs2544600 (SEQ ID NO: 11) has a single nucleotide variation C in place of ancestral T at position 26 (T26G) of SEQ ID NO: 12 (ancestral sequence) from Chromosome 5 of Homo sapiens.

Methods for detecting a SNP in an subject are known in the art (see e.g., Dynamic allele-specific hybridization, DASH; molecular beacons; SNP microarrays; Restriction fragment length polymorphism, RFLP; tetra-primer ARMS-PCR; Flap endonuclease, FEN; primer extension; Taq DNA polymerase 5′-nuclease activity in a TaqMan assay; oligonucleotide ligation assay; single strand conformation polymorphism; temperature gradient gel electrophoresis;

denaturing high performance liquid chromatography; high resolution melting analysis; DNA mismatch-binding proteins; SNPlex; or next-generation sequencing technologies such as pyrosequencing). Except as otherwise noted herein, therefore, detection of a SNP in a subject can be carried out in accordance with such processes.

A SNP can be detected in a sample of a subject. A sample can be a biological sample from a subject. A sample can include cells of a subject. For example, a sample can be a solid tissue sample. As another example, a sample can be a buccal swab.

As described herein, a SNP array can be paired with another analytical tool (i.e., a complete exome analysis) to enhance the ability to explore the concept of a genetic cause for pelvic organ prolapse surgery failure.

Subject

Methods described herein can be generally performed on a subject in need thereof. A subject in need of methods or compositions described herein can be a subject having, diagnosed with, suspected of having, or at risk for developing female pelvic organ prolapse. A female pelvic organ prolapse can include, but is not limited to, uterine prolapse or female genital prolapse. For the purposes of the present disclosure, POP will often be recited but one of ordinary skill will understand that such disclosure can apply equally to any female pelvic organ prolapse to the extent these terms are not synonymous or co-extensive.

A subject in need of methods or compositions described herein can be a subject who is a candidate for female pelvic organ prolapse surgery (e.g., sacrocolpopexy). A subject in need of methods or compositions described herein can be a subject who is a candidate for sacrocolpopexy. A subject in need of methods or compositions described herein can be a subject who is a candidate for laparoscopic sacrocolpopexy. A subject in need of methods or compositions described herein can be a subject who is a candidate for robotic-assisted laparoscopic sacrocolpopexy protocols. A subject in need of methods or compositions described herein can be a subject who is having, diagnosed with, suspected of having, or at risk for developing POP or who is a candidate for sacrocolpopexy, laparoscopic sacrocolpopexy, or robotic-assisted laparoscopic sacrocolpopexy protocols.

POP is understood in the art to be any descent of the anterior vaginal wall (aka cystocele or urethrocele), the vaginal apex (aka uterine or vaginal vault prolapse), or the posterior vaginal wall (aka rectocele, perineocele), or all of these. Symptoms that can be commonly associated with POP include pelvic heaviness, vaginal bulging, incomplete bowel or bladder emptying, needing to splint the posterior vaginal wall or perineum to defecate, or discomfort during sexual intercourse. Risk factors include vaginal childbirth, frequent increases in intra-abdominal pressure (such as occurs with heavy lifting or chronic constipation), aging, or genetic predisposition to connective tissue abnormalities.

A determination of the need for treatment will typically be assessed by a history or physical exam consistent with the disease or condition at issue. Diagnosis of the various conditions treatable by the methods described herein is within the skill of the art. POP is understood in the art to be any descent of the anterior vaginal wall (aka cystocele or urethrocele), the vaginal apex (aka uterine or vaginal vault prolapse), or the posterior vaginal wall (aka rectocele, perineocele), or all of these. Symptoms that can be commonly associated with POP include pelvic heaviness, vaginal bulging, incomplete bowel or bladder emptying, needing to splint the posterior vaginal wall or perineum to defecate, or discomfort during sexual intercourse. The etiology of POP can be multifactorial and complicated. The main risk factors can be vaginal childbirth, frequent increases in intra-abdominal pressure (such as occurs with heavy lifting or chronic constipation), aging, or genetic predisposition to connective tissue abnormalities.

A prolapse can be graded via the Baden-Walker System, Shaw's System, or the Pelvic Organ Prolapse Quantification (POP-Q) System. A subject in need can have mild, moderate, or severe prolapse under one or more of these systems. A subject in need can have mild prolapse under one or more of these systems. A subject in need can have moderate prolapse under one or more of these systems. A subject in need can have severe prolapse under one or more of these systems.

The subject can be mammalian subject, such as a human. A subject can be an individual subject. A subject can be one or more subjects. A subject can be a plurality of subjects. A subject can be a subject population.

Female Pelvic Organ Prolapse Surgery

Also provided are diagnostic methods and devices for use in conjunction with treatment of female pelvic organ prolapse in a subject in need. A subset of female subjects can be at increased risk for early overt failure following female pelvic organ prolapse surgery (e.g., sacrocolpopexy). A SNP described herein can identify a subject in this at-risk grouping.

Female pelvic organ prolapse surgery is known in the art. Female pelvic organ prolapse surgeries include, but are not limited to, repair of the prolapsed bladder (cystocele) or urethra (urethrocele); removal of the uterus (hysterectomy); repair of the rectum (rectocele) or small bowel (enterocele); repair of the vaginal wall (vaginal vault suspension); or closure of the vagina (colpocleisis or vaginal obliteration). Except as otherwise noted herein, therefore, the compositions and methods of the present disclosure can be carried out in accordance with such processes.

Female pelvic organ prolapse surgery can be a sacrocolpopexy protocol. Sacrocolpopexy protocols are well known in the art. Laparoscopic sacrocolpopexy protocols are well known in the art. Robotic-assisted laparoscopic sacrocolpopexy protocols are well known in the art. Except as otherwise noted herein, therefore, the compositions and methods of the present disclosure can be carried out in accordance with such processes.

The female pelvic organ prolapse surgery can be a method that involves extensive dissection in the vesicovaginal and rectovaginal spaces. A pre-formed polypropylene “Y-mesh” can be attached to the full length and width of the anterior vaginal wall (down to the level of the trigone) and to the full length and width of the posterior vaginal wall (down to the level of the perineal body). Using this technique, the inventors have reported surgical cure rates of 97% at one year, with few failures typically occurring in the distal anterior or posterior segments. The compositions and methods of the present disclosure can be carried out in accordance with the method for treating female pelvic organ prolapse described above.

Each of the states, diseases, disorders, or conditions, described herein, as well as others, can benefit from compositions or methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

When a SNP described herein is identified, a subject or attending physician can alter a surgical protocol, select a therapeutic approach other than surgery, select a therapeutic approach other than sacrocolpopexy; delay a surgical approach, forgo a surgical approach, or increasing a TGFβ level in the subject.

Device

Also provided is a device for use in detecting a SNP described herein. Such a device can detect one of more SNPs described herein. A device as described herein can be contacted with a biological sample so as to detect presence of one or more SNPs described herein. A device as described herein can be contacted with a biological sample so as to detect presence of one or more SNPs described herein among alleles within or between populations. A combination of one or more SNPs described herein and a SNP array can allows SNPs to be used as markers for genetic basis for failure of a female pelvic organ prolapse surgery (e.g., sacrocolpopexy).

A SNP array can be a type of DNA microarray used to detect one or more polymorphisms within a population. Devices for detection of SNPs are understood in the art (see e.g., LaFramboise 2009 Nucleic Acid Res 37(13), 4181-4193). One of ordinary skill in the art can adapt conventional SNP detection devices for specificity with respect to one or more SNPs described herein.

A device can include an array (e.g., a microarray) for detection of one of more SNPs described herein. A SNP array is understood as a convergence of polynucleotide hybridization, fluorescence microscopy, and solid surface polynucleotide capture. A SNP array device can include: one or more labeled allele-specific oligonucleotide (ASO) probes; or a detection system that records or interprets the hybridization signal. The ASO probes can be specific for one or more SNPs described herein. The array device can be contacted with a sample including fragmented nucleic acid sequences that could include one or more SNPs described herein, which can be labeled with a fluorescent dye. Hybridization of a fragmented nucleic acid sequence and an ASO probe can result in a hybridization signal recorded or interpreted by the detection system.

An array device can include one or more DNA detection spots. Detection of one or more SNPs described herein can be on a dedicated array device or included with one or more related or unrelated DNA detection spots. For example, an array can contain one or a few probes (e.g., 1, 2, 3, 4, 5, 6, 7, 8, 9, or 10 probes) up to several million probes. Each DNA spot (e.g., an allele-specific oligonucleotide (ASO) probes; a SNP spot) can contain picomoles (10-12 moles) of a specific sequence (e.g., a sequence including a SNP described herein), known as probes (or reporters or oligos). These sequences can be used to hybridize a polynucleotide sample (e.g., cDNA or cRNA) (i.e., target) under high-stringency conditions. After washing off non-specific bonding sequences, only strongly paired strands will remain hybridized. Probe-target hybridization can be detected or quantified by detection of fluorophore-, silver-, or chemiluminescence-labeled targets.

A SNP chip can be described by the number of SNP positions assayed. Two probes can be used for each SNP position to detect both alleles. If only one probe were used, experimental failure may be indistinguishable from homozygosity of the non-probed allele.

Probes (e.g., ASO probes) can be synthesized and then attached via surface engineering to a solid surface by a covalent bond to a chemical matrix (e.g., via epoxy-silane, amino-silane, lysine, polyacrylamide or others). The matrix surface can be glass or a silicon chip or a microscopic bead (e.g., polystyrene bead). A microarray can be constructed by direct synthesis of oligonucleotide probes on solid surfaces. A microarray can be a spotted microarray. A microarray can be an oligonucleotide microarray. A microarray can be a one-color microarray. A microarray can be a two-color microarray. A microarray can be a one-channel microarray. A microarray can be a two-channel microarray. A single-dye system can have the advantage that an aberrant sample cannot affect the raw data derived from other samples, because each array chip is exposed to only one sample (as opposed to a two-color system in which a single low-quality sample may impinge on overall data precision even if the other sample was of high quality).

Exemplary commercially available microarray providers include Agilent (Dual-Mode platform), Eppendorf (DualChip platform for colorimetric Silverquant labeling), TeleChem International (Arrayit), Affymetrix (Gene Chip), Illumina (Bead Chip), Agilent (single-channel arrays), Applied Microarrays (CodeLink arrays), and Eppendorf (DualChip & Silverquant).

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a device (e.g., a microarray) to detect one or more SNP described herein or a sample collection system. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials.

Instructions may be printed on paper or other substrate, or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Green and Sambrook 2012 Molecular Cloning: A Laboratory Manual, 4th ed., Cold Spring Harbor Laboratory Press, ISBN-10: 1605500569; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### EXAMPLE 1

### Standardized Robotic Sacrocolpopexy

A surgical method for treating POP involves extensive dissection in the vesicovaginal and rectovaginal spaces. A pre-formed polypropylene “Y-mesh” can be attached to the full length and width of the anterior vaginal wall (down to the level of the trigone) and to the full length and width of the posterior vaginal wall (down to the level of the perineal body). Using standardized robotic techniques, the surgical cure rates using this technique were 97% at one year, with few failures typically occurring in the distal most anterior or posterior segments.

But a small group of patients can experience early objective overt failures despite having the extensive procedure described above in the early post-operative period. These failures have not been explained by differing surgical techniques, poor adherence to post-operative restrictions, or complicated peri-operative courses. In other words, the failures in this small group were clinically difficult to explain.

The objective of the following studies were to determine whether a genetic basis existed for the early overt surgical failures seen within this ‘extreme phenotype’ group.

### EXAMPLE 2

### SNP Identification in Subjects With Early Surgical Failure

The following example shows identification of SNPs associated with risk of failure in female pelvic organ prolapse surgery.

For the purposes of this study, ‘early overt failure’ was defined to be development of stage III or IV prolapse based on the pelvic organ prolapse quantification system (POP-Q) occurring in more than one compartment within six months of robotic-assisted laparoscopic sacrocolpopexy surgery. The clinical records were reviewed to find any patients who were found to have stage II or greater prolapse after undergoing the standardized robotic sacrocolpopexy with one of two attendings between 2005 and 2013. The medical records of this group were reviewed to identify those patients who required downstream surgical or non-surgical pelvic organ prolapse treatments. Resultant potential cases were then reviewed by urogynecology attendings to select only those patients deemed true clinical outliers.

10 subjects were selected who experienced early overt robotic-assisted laparoscopic sacrocolpopexy surgical failure and thus made up the “extreme phenotype” group. 40 controls were randomly selected from a research database that included greater than 500 patients who underwent robotic-assisted laparoscopic sacrocolpopexy with polypropylene mesh during the same time period and had documented objective and subjective surgical success at 12 months or more. Because all 10 subjects were Caucasian, eligible controls were Caucasian as well. In addition, eligible controls had to have experienced uneventful perioperative courses, and had to have documented objective/subjective surgical success at 12 months or more. Potential control subjects were selected at random (using a random number generator) from the database and contacted (one at a time) regarding study participation. Those who agreed were mailed buccal swab kits and consent forms. Each time a potential control subject declined enrollment, another was chosen at random from the database. This process was repeated until 40 controls were enrolled. Demographics and peri-operative details were compared between cases and controls.

All cases and controls underwent the standardized technique for robotic assisted laparoscopic sacrocolpopexy by one of two surgeons at a single center. The details of the surgical technique have been previously published (Culligan 2014; Salamon, 2013). Briefly, an extensive dissection was performed in the vesicovaginal and rectovaginal spaces to the level of the trigone and perineum respectively. A pre-formed polypropylene “Y-mesh” was attached to the full length and width of the anterior vaginal wall (down to the level of the trigone) and to the full length and width of the posterior vaginal wall (down to the level of the perineal body) using interrupted polytetrafluroethelyene sutures (Gore-Tex, WL Gore, Flagstaff Ariz.). The mesh is then attached to the anterior longitudinal ligament of the sacrum using permanent suture of the surgeon's choice.

DNA from the 10 subjects and 40 controls was isolated from buccal swabs and genotyped on a single nucleotide polymorphism (SNP) array that contains probes for approximately 262,000 markers (Nspl 250K SNP array, Affymetrix, Santa Clara, Calif.). Statistical analyses were performed using a statistical software package commonly used in genetics studies (SVS, GoldenHelix, Bozeman, Mont.). Tests performed were the Correlation/Trend test, the Cochran-Armitage test and logistic regression. Genotype models (D minor allele, d major allele) were basic allele (D vs d), Genotypic (DD vs dd vs Dd), Additive (dd−>Dd−>DD), Dominant (DD and Dd vs. dd), and Recessive (DD vs (Dd and dd)). Association analysis and quality control filtering was performed using GoldenHelix SVS and p-values were adjusted for multiple testing using the False Discovery Rate (FDR) to control for the expected proportion of incorrectly rejected null hypotheses (“false discoveries”). Baseline demographic and clinical descriptors for the cases and controls were compared using Chi square, Mann Whitney U, and t-tests, and principal component analysis testing for genotype stratification was performed to identify any patients as outliers.

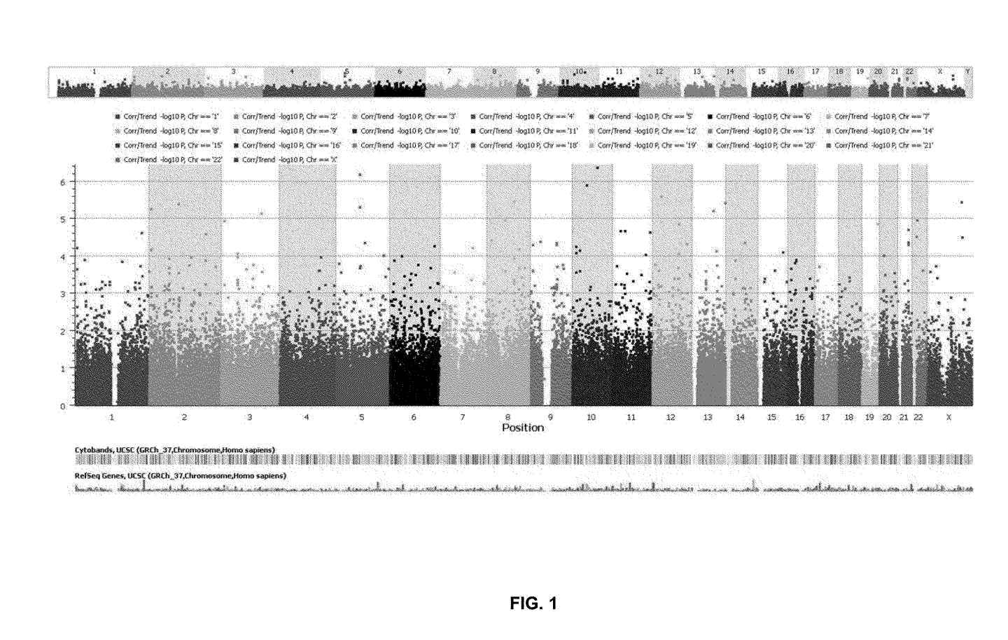

Candidate genetic loci identified by the SNP array based genome-wide association analyses (see e.g., FIG. 1) were further investigated by specifically evaluating the sequence around the SNP using whole exome sequence data from the same sample set. Whole exome sequencing was performed using the Ion AmpliSeq Exome Solution (Thermo Fischer Scientific Inc., Waltham, Mass.) as recommended by the supplier with 2 samples per P1 chip. Exome sequences were mapped to the Human Genome Reference Sequence (GRCh37) using Bowtie2 version 2.1.0 and processed with samtools version 0.1.19-44428cd. Variants were compared between cases and controls and called with VarScan 2.3.5. Effects of variants on gene structure were estimated with snpEFF 3.5, and the protein function effect analysis was estimated with the bioinformatic tool PROVEAN. Variants with putative functional significance identified by next-generation sequencing were independently verified using TaqMan quantitative real time PCR based allelic discrimination, as recommended by the supplier (Thermo Fischer Scientific Inc., Waltham, Mass.).

To evaluate the robustness of the findings and to verify the rare nature of the SNPs in question, DNA from the cases was further compared to DNA from participants in the 1000 Genomes Project. There are 379 Caucasians within that database, so only that group was used as comparison DNA to the cases.

All data used in the study were deposited in NCBI's Gene Expression Omnibus and are accessible through GEO Series accession number GSE63236.

Initial analysis via SNP array yielded a SNP (dbSNP ID rs171821, SEQ ID NO: 1) located near the ZFYVE16 gene (also known as Endofin) on chromosome 5 that was associated with the group of cases but not the controls and correlation/trend testing on the basic allele model yielded a false discovery rate adjusted p-value of 0.046 (odds ratio 45.2, 95% confidence interval 5.06-403). In addition, three other SNPs were found within the ZFYVE16 gene near dbSNP ID rs171821 (SEQ ID NO: 1) with raw p-values of <0.001, but after false discovery rate adjustment these 3 SNPs were not statistically significantly associated. These 3 SNP IDs were dbSNP IDs rs1423113 (SEQ ID NO: 3), rs16877757 (SEQ ID NO: 5), and rs259043 (SEQ ID NO: 7). Although the these three SNPs (rs1423113 (SEQ ID NO: 3), rs16877757 (SEQ ID NO: 5), and rs259043 (SEQ ID NO: 7)) did not hold up against false discovery rate control statistical test, it is believed that they play a role in prolapse surgical failures of other varieties (e.g., failure after native tissue repair).

These findings prompted performance of an exome analysis in the region of the ZFYVE16 gene.

This exome analysis yielded the SNP dbSNP ID rs249038 (G/A) (SEQ ID NO: 9), which was present in 6 of 10 cases and none of the controls (Fisher two-tailed p=0.02). This SNP is rare in European populations and is a heterozygous (G/A) missense mutation that results in formation of serine rather than glycine by the Endofin gene. The remaining cases and all controls expressed the expected homozygous (G/G) pattern. Genotypes for this locus were confirmed with 100% concordance using TaqMan allelic discrimination. A significant difference was recognized when data from the 1000 Genomes Project was used to compare the cases to 379 Caucasians (p<0.001). This change from glycine to serine was predicted to be deleterious by the bioinformatic tool Protein Variation Effect Analyzer (PROVEAN).

All of the surgical procedures for cases as well as controls were uncomplicated and perioperative courses were unremarkable. No cases or controls required conversion from laparoscopy to laparotomy and there were no peri-operative complications in either group. The mean estimated blood loss for the cases and controls was 52.5 (±53.3) ml and 55.5 (±50.1) ml, respectively (p=0.87). Average operative time for cases and controls were 167 (±27.9) and 151 (±30.7) minutes, respectively (p=0.15). All 50 patients were discharged home the day after surgery with no re-admissions. There were no baseline demographic or clinical differences between the cases and controls, and principal component analysis testing for genotype stratification did not identify any patients as outliers (see e.g., Table 1).

TABLE 1
Characteristics and Demographics of the Study Population

| | Cases (n = | Controls (n = | |
|---|---|---|---|
| Characteristic | 10) | 40) | P value |
| Age - yr^{a} | 58.5 ± 5.5 | 59.9 ± 8.2 | 0.61 |
| BMI^{a} | 26.6 ± 3.1 | 24.9 ± 4.4 | 0.26 |
| Parity^{b} | 2.5 (1-4) | 2.0 (1-4) | 0.76 |
| Tobacco use-no. (%) | 1 (10) | 2 (5) | 0.50 |
| Menopausal-no. (%) | 8 (80) | 30 (75) | 0.39 |
| Hormone replacement therapy-no. (%) | 1 (10) | 4 (10) | 0.47 |
| Prior hysterectomy-no. (%) | 2 (20) | 8 (20) | 0.48 |
| Prior POP surgery-no. (%) | 2 (20) | 4 (10) | 0.22 |
| POP-Q leading edge pre-op-cm.^{b} | 3.5 (0-10) | 1.75 (−1-10) | 0.18 |
| Total | 10 | 40 | |

^{a}mean ± (standard deviation)
^{b}median (range)

Results showed that a SNP located near the ZFYVE16 gene (rs171821, SEQ ID NO: 1) was associated with the group of cases but not the controls. The correlation/trend test on the basic allele model had an FDR-adjusted p-value of 0.046. In addition, in the same test, association p-values of three other SNPs near dbSNP ID rs171821 on Chromosome 5 and also in the ZFYVE16 gene were found in the top 20 ranked SNPs with raw p<0.0001 but did not pass FDR adjustment (with dbSNP IDs rs1423113 (SEQ ID NO: 3), rs16877757 SEQ ID NO: 5), and rs259043 (SEQ ID NO: 7)).

The findings indicate that a candidate gene on chromosome 5, ZFYVE16 (aka Endofin), is linked to early recurrence of prolapse. The initial analyses identified four candidate SNPs near Endofin, one of which was found to be truly statistically unique to the group of cases. Upon exome sequence analysis of this region, the variant known as dbSNP ID rs249038 (G/A at position 26) (SEQ ID NO: 9) was identified and determined to cause a rare missense mutation predicted to be deleterious.

Although the other three SNPs found did not hold up against false discovery rate control statistical test, it is believed that the other three SNPs play a role in prolapse surgical failures of other varieties (e.g., failure after native tissue repair).

The locations of the SNPs found on or near the Endofin gene support the importance of the findings because Endofin facilitates Transforming Growth Factor-beta (TGF-β) as a scaffold protein.

Sequence Listing

dbSNP ID rs171821 with single nucleotide T
variation, Homo sapiens,
SEQ ID NO: 1
GTATTTTCTTTACCCAGGTTACTTA[T]GAAAAGTGAATAGGTTTGGG
AGTTC
Ancestral sequence of dbSNP ID rs171821 with C
allele, Homo sapiens
SEQ ID NO: 2
GTATTTTCTTTACCCAGGTTACTTA[C]GAAAAGTGAATAGGTTTGGG
AGTTC
dbSNP ID rs1423113 with single nucleotide C
variation, Homo sapiens
SEQ ID NO: 3
AGATGAAACTAGGTTGTCCATATTG[C]AGCTGGATTATGGGATTGGC
TACGA
Ancestral sequence of dbSNP ID rs1423113 with A
allele, Homo sapiens
SEQ ID NO: 4
AGATGAAACTAGGTTGTCCATATTG[A]AGCTGGATTATGGGATTGGC
TACGA
dbSNP ID rs16877757 with single nucleotide G
variation, Homo sapiens
SEQ ID NO: 5
ACTAACAAGTAGAATGTTTAATTTC[G]CTTCTCTCACTTGAATTTCA
GTTCT
Ancestral sequence of dbSNP ID rs16877757 with A
allele, Homo sapiens
SEQ ID NO: 6
ACTAACAAGTAGAATGTTTAATTTC[A]CTTCTCTCACTTGAATTTCA
GTTCT
dbSNP ID rs259043 (reverse strand) with single
nucleotide G variation, Homo sapiens
SEQ ID NO: 7
ATGATCCAAACTTTGCCAAGGATAC[G]TTTTCGTCAATATTTGATTT
GACAC
Ancestral sequence of dbSNP ID rs259043 with T
allele, Homo sapiens
SEQ ID NO: 8
ATGATCCAAACTTTGCCAAGGATAC[T]TTTTCGTCAATATTTGATTT
GACAC
dbSNP ID rs249038 with single nucleotide A
variation, Homo sapiens
SEQ ID NO: 9
GATCATTTTGCTTCTTGAAGGTGAA[A]GCTTTCATCCTGTTACATTT
GTCCT
Ancestral sequence of dbSNP ID rs249038 with G
allele, Homo sapiens
SEQ ID NO: 10
GATCATTTTGCTTCTTGAAGGTGAA[G]GCTTTCATCCTGTTACATTT
GTCCT
dbSNP ID rs2544600 with single nucleotide C
variation, Homo sapiens
SEQ ID NO: 11
ACTGTCAGAGAACAACAGAATGATA[C]CAGTTCTGAATTACAAAATA
GAGAA
Ancestral sequence of dbSNP ID rs2544600 with T
allele, Homo sapiens
SEQ ID NO: 12
ACTGTCAGAGAACAACAGAATGATA[T]CAGTTCTGAATTACAAAATA
GAGAA

### REFERENCES

Culligan P , Gurshumov E, Lewis C, et al. Subjective and objective results 1 year after robotic sacrocolpopexy using a lightweight Y-mesh. Int Urogynecol J. 2014 June; 25(6):731-35.
Salamon C G, Lewis C, Priestley J, Gurshumov E, et al. Prospective study of an ultra-lightweight polypropylene Y mesh for robotic sacrocolpopexy. Int Urogynecol J. 2013; 24(8):1371-5.
Langmead B, Salzberg S. Fast gapped-read alignment with Bowtie 2. Nature Methods. 2012; 9(4):357-359.
Li H, Handsaker B, Wysoker A, et al. The Sequence Alignment/Map format and SAMtools. Bioinformatics. 2009; 25(16):2078-9.
Koboldt D C, Chen K, Wylie T, et al. VarScan: variant detection in massively parallel sequencing of individual and pooled samples. Bioinformatics. 2009 Sep; 25(17):2283-5.
Cingolani P, Platts A, Wang le L, et al. A program for annotating and predicting the effects of single nucleotide polymorphisms, SnpEff: SNPs in the genome of Drosophila melanogaster strain w1118; iso-2; iso-3. Fly (Austin). 2012 Apr-Jun; 6(2):80-92.
Choi Y, Sims G E, Murphy S, et al. Predicting the Functional Effect of Amino Acid Substitutions and Indels. PLoS ONE 2012; 7(10): e46688.

McVean G A, Abecasis G R, Auton A, et al. An integrated map of genetic variation from 1,092 human genomes. Nature. 2012 Nov; 491(7422):56-65.
Edgar R, Domrachev M, Lash AE. Gene Expression Omnibus: NCBI gene expression and hybridization array data repository. Nucleic Acids Res. 2002 Jan 1; 30(1):207-10.
FDA. Urogynecologic Surgical Mesh: Update on the safety and effectiveness of transvaginal placement or pelvic organ prolapse. E. F.a.D. Administration, Editor 2011 July.
Cartwright R, Kirby A, Tikkinen K, et al. Systematic review and metaanalysis of genetic association studies of urinary symptoms and prolapse in women. Am J Obstet Gynecol. 2015 Feb; 212(2):199-201.
Kobak W, Lu J, Hardart A, et al. Expression of lysyl oxidase and transforming growth factor β2 in women with severe pelvic organ prolapse. Journal of Reproductive Medicine. 2005 Nov; 50(11):827-831.
Cook J R, Carta L, Galatioto J, et al. Cardiovascular manifestations in Marfan syndrome and related diseases; multiple genes causing similar phenotypes. Clinical Genetics. 2015 Jan; 87(1):11-20.
Carley M E, Schaffer J. Urinary incontinence and pelvic organ prolapse in women with Marfan or Ehlers Danlos syndrome. Am J Obstet Gynecol. 2000 May; 182(5):1021-3.
Qi X Y, Hong L, Guo F Q, et al. Expression of transforming growth factor-beta 1 and connective tissue growth factor in women with pelvic organ prolapse. Saudi Med J. 2011 May; 32(5):474-8.
Li D, Lewinger J P, Gauderman W J, et al. Using extreme phenotyping sampling to identify the rare causal variants of quantitative traits in association studies. Genet Epidemiol. 2011 Dec; 35(8):790-9.
Culligan P, Murphy M, Blackwell L, et al. Long-term success of abdominal sacral colpopexy using synthetic mesh. Am J Obstet Gynecol. 2002; 187(6):1473-82.

Wu J M, Hundley A F, Fulton R G, Myers E R. Forecasting the prevalence of pelvic floor disorders in US women: 2010 to 2050. Obstet Gynecol 2009;114:1278-83

Bump R C, Mattiasson A, Bo K, Brubaker L P, DeLancey J O, Klarskov P, et al. The standardization of terminology of female pelvic organ prolapse and pelvic floor dysfunction. Anm J Obstet Gynecol 1996;175:10-17Tseng L H, Chen I, Lin Y H, Chen M Y, Lo T S, Lee C L. Genome-based expression profiles study for the pathogenesis of pelvic organ prolapse: an array of 33 genes model. Int Urogynecol J 2010; 21:79-84
Hundley A F, Yuan L, and Visco A G. Gene expression in the rectus abdominus muscle of patients with and without pelvic organ prolapse. Am J Obstet Gynecol 2008;198:220.e1-220.e7.
Allen-Brady K, Norton P A, Farnham J M, Teerlink C, Cannon-Albright L A. Significant Linkage Evidence for a Predisposition Gene for Pelvic Floor Disorders on Chromosome 9q21. The American Journal of Human Genetics 2009; 84:678-82.
Kluivers K B, Dijkstra J R, Hendriks J C M, Lince S L, Vierhout M E, van Kempen L C L. COL3A1 2209G>A is a predictor of pelvic organ prolapse. Int Urogynecol J 2009; 20:1113-8

Sun M J, Cheng W L, Wei Y H, Kuo C L, Sun S, Tsai H D, Lin H M, Liu C S. Low copy number and high 4977 deletion of mitochondrial DNA in uterosacral ligaments are associated with pelvic organ prolapse progression. Int Urogynecol J 2009;20:867-72.

Skorupski P, Kro'I J, Julia Stare J, Adamiak A, Jankiewicz K, Rechberger T. An α-1 chain of type I collagen Sp1-binding site polymorphism in women suffering from stress urinary incontinence. Am J Obstet Gynecol 2006;194:346-50
Nygaard I E, McCreery R, Brubaker L, et al. Abdominal sacrocolpopexy: a comprehensive review. Obstet Gynecol. 2004 Oct; 104(4):805-23.
Culligan P J, Gurshumov E, Lewis C, et al. Subjective and objective results 1 year after robotic sacrocolpopexy using a lightweight Y-mesh. IntUrogynecol J. 2013 Oct; online: DOI 10.1007/s00192-013-2265-x
Chen Y G, Wang Z, Ma J, et al. Endofin, a FYVE domain protein, interacts with Smad4 and facilitates transforming growth factor-beta signaling. J Biol Chem. 2007 Mar; 282:9688-9695

1. A method for diagnosing an increased risk of failure in a female pelvic organ prolapse surgery, the method comprising:
detecting at least one single nucleotide polymorphism (SNP) in a subject in need; and
correlating presence of the SNP with an increased risk of failure in a female pelvic organ prolapse surgery;
wherein the at least one SNP is selected from the group consisting of:
a single nucleotide variation T at position 26 of SEQ ID NO: 2;
a single nucleotide variation C at position 26 of SEQ ID NO: 4;
a single nucleotide variation G at position 26 of SEQ ID NO: 6;
a single nucleotide variation G at position 26 of SEQ ID NO: 8;
a single nucleotide variation A at position 26 of SEQ ID NO: 10; and
a single nucleotide variation C at position 26 of SEQ ID NO: 12.

2. The method of claim 1, wherein the SNP is selected from the group consisting of:
a single nucleotide variation T at position 26 of SEQ ID NO: 2; and
a single nucleotide variation A at position 26 of SEQ ID NO: 10.

3. The method of claim 2, further comprising a SNP selected from the group consisting of:
a single nucleotide variation C at position 26 of SEQ ID NO: 4;
a single nucleotide variation G at position 26 of SEQ ID NO: 6; and
a single nucleotide variation G at position 26 of SEQ ID NO: 8.

4. The method of claim 1, wherein the SNP is present in a sequence selected from the group consisting of SEQ ID NO: 1 (dbSNP ID rs171821); SEQ ID NO: 3 (dbSNP ID rs1423113); SEQ ID NO: 5 (dbSNP ID rs16877757); SEQ ID NO: 7 (dbSNP ID rs259043); SEQ ID NO: 9 (dbSNP ID rs249038); and SEQ ID NO: 11 (dbSNP ID rs2544600).

5. The method of claim 4, wherein the SNP is present in a sequence selected from the group consisting of SEQ ID NO: 1 (dbSNP ID rs171821); and SEQ ID NO: 9 (dbSNP ID rs249038).

6. The method of claim 5, further comprising a SNP is present in a sequence selected from the group consisting of SEQ ID NO: 3 (dbSNP ID rs1423113); SEQ ID NO: 5 (dbSNP ID rs16877757); and SEQ ID NO: 7 (dbSNP ID rs259043).

7. The method of claim 1, wherein detecting the SNP comprises detecting a plurality of SNPs.

8. The method of claim 1, wherein the subject has, is diagnosed with, is suspected of having, or is at risk for developing female pelvic organ prolapse.

9. The method of claim 1, further comprising:
providing a biological sample from the subject;
wherein detecting the SNP comprises detecting the SNP in the biological sample.

10. The method of claim 9, wherein the sample comprises a buccal swab.

11. The method of claim 1, further comprising:
if the SNP is detected, altering a surgical protocol, selecting a therapeutic approach other than surgery, selecting a therapeutic protocol other than sacrocolpopexy; delaying a surgical protocol, or forgoing a surgical protocol.

12. The method of claim 1, further comprising an exome analysis.

13. The method of claim 1, wherein the SNP is detected by Dynamic allele-specific hybridization, DASH; molecular beacons; SNP microarrays;
Restriction fragment length polymorphism, RFLP; tetra-primer ARMS-PCR; Flap endonuclease, FEN; primer extension; Taq DNA polymerase 5′-nuclease activity in a TaqMan assay; oligonucleotide ligation assay; single strand conformation polymorphism; temperature gradient gel electrophoresis; denaturing high performance liquid chromatography; high resolution melting analysis; DNA mismatch-binding proteins; SNPlex; or pyrosequencing.

14. The method of claim 1, wherein the SNP is detected by an array.

15. The method of claim 1, wherein the SNP is detected by an array comprising:
a labeled allele-specific oligonucleotide (ASO) probe specific for
(i) a SNP selected from the group consisting of a single nucleotide variation T at position 26 of SEQ ID NO: 2; a single nucleotide variation C at position 26 of SEQ ID NO: 4; a single nucleotide variation G at position 26 of SEQ ID NO: 6; a single nucleotide variation G at position 26 of SEQ ID NO: 8; a single nucleotide variation A at position 26 of SEQ ID NO: 10; and a single nucleotide variation C at position 26 of SEQ ID NO: 12; or
(ii) a SNP present in SEQ ID NO: 1 (dbSNP ID rs171821), SEQ ID NO: 3 (dbSNP ID rs1423113), SEQ ID NO: 5 (dbSNP ID rs16877757), SEQ ID NO: 7 (dbSNP ID rs259043), SEQ ID NO: 9 (dbSNP ID rs249038), or SEQ ID NO: 11 (dbSNP ID rs2544600); and
a detection system that records or interprets a hybridization signal between the ASO probe and a polynucleotide sequence from a biological sample of the subject.

16. The method of claim 16, further comprising contacting a biological sample of the subject and the array.

17. The method of claim 1, further comprising detecting a level of TGFβ in the subject, wherein detecting the decreased level of TGFβ in the subject is associated with increased risk of failure in a female pelvic organ prolapse surgery.

18. The method of claim 1, further comprising:
if the SNP is detected, altering a surgical protocol, selecting a therapeutic approach other than surgery, selecting a therapeutic protocol other than sacrocolpopexy; delaying a surgical protocol, forgoing a surgical protocol, or increasing a TGFβ level in the subject.

19. A device for detection of a single nucleotide polymorphism (SNP) associated with increased risk of failure in a female pelvic organ prolapse surgery, the device comprising:
one or more labeled allele-specific oligonucleotide (ASO) probes specific for
(i) a SNP selected from the group consisting of a single nucleotide variation T at position 26 of SEQ ID NO: 2; a single nucleotide variation C at position 26 of SEQ ID NO: 4; a single nucleotide variation G at position 26 of SEQ ID NO: 6; a single nucleotide variation G at position 26 of SEQ ID NO: 8; a single nucleotide variation A at position 26 of SEQ ID NO: 10; and a single nucleotide variation C at position 26 of SEQ ID NO: 12; or
(ii) a SNP present in SEQ ID NO: 1 (dbSNP ID rs171821), SEQ ID NO: 3 (dbSNP ID rs1423113), SEQ ID NO: 5 (dbSNP ID rs16877757), SEQ ID NO: 7 (dbSNP ID rs259043), SEQ ID NO: 9 (dbSNP ID rs249038), or SEQ ID NO: 11 (dbSNP ID rs2544600); and
a detection system that records or interprets a hybridization signal between the ASO probe and a polynucleotide sequence from a biological sample of the subject.

20. The device of claim 19, comprising an array, the array comprising a matrix and a plurality of detection spots on or in the matrix, each detection spot comprising a unique ASO probe.

21. The device of claim 20, wherein the array device consists essentially of detection spots comprising unique ASO probe specific for a SNP associated with increased risk of failure in a female pelvic organ prolapse surgery.

22. The device of claim 20, wherein the array device comprises (i) detection spots comprising unique ASO probe specific for a SNP associated with increased risk of failure in a female pelvic organ prolapse surgery and (ii) detection spots not so associated.


SEQUENCE LISTING (12 sequences)

SEQ ID NO: 1 - DNA, 51 bp, Homo sapiens
gtattttctttacccaggttacttatgaaaagtgaataggtttgggagttc

SEQ ID NO: 2 - DNA, 51 bp, Homo sapiens
gtattttctttacccaggttacttacgaaaagtgaataggtttgggagttc

SEQ ID NO: 3 - DNA, 51 bp, Homo sapiens
agatgaaactaggttgtccatattgcagctggattatgggattggctacga

SEQ ID NO: 4 - DNA, 51 bp, Homo sapiens
agatgaaactaggttgtccatattgaagctggattatgggattggctacga

SEQ ID NO: 5 - DNA, 51 bp, Homo sapiens
actaacaagtagaatgtttaatttcgcttctctcacttgaatttcagttct

SEQ ID NO: 6 - DNA, 51 bp, Homo sapiens
actaacaagtagaatgtttaatttcacttctctcacttgaatttcagttct

SEQ ID NO: 7 - DNA, 51 bp, Homo sapiens
atgatccaaactttgccaaggatacgttttcgtcaatatttgatttgacac

SEQ ID NO: 8 - DNA, 51 bp, Homo sapiens
atgatccaaactttgccaaggatactttttcgtcaatatttgatttgacac

SEQ ID NO: 9 - DNA, 51 bp, Homo sapiens
gatcattttgcttcttgaaggtgaaagctttcatcctgttacatttgtcct

SEQ ID NO: 10 - DNA, 51 bp, Homo sapiens
gatcattttgcttcttgaaggtgaaggctttcatcctgttacatttgtcct

SEQ ID NO: 11 - DNA, 51 bp, Homo sapiens
actgtcagagaacaacagaatgataccagttctgaattacaaaatagagaa

SEQ ID NO: 12 - DNA, 51 bp, Homo sapiens
actgtcagagaacaacagaatgatatcagttctgaattacaaaatagagaa